(19) United States
(12) Patent Application Publication
(10) Pub. No.: US 2020/0176110 A1
(43) Pub. Date: Jun. 4, 2020

(54) Personal Health Management System

(71) Applicant: Innovative Simulation and Patient Involvement Resources Global LLC, Lake Forest, CA (US)

(72) Inventor: Lori MARSHALL, Lake Forest, CA (US)

(21) Appl. No.: 16/698,097

(22) Filed: Nov. 27, 2019

Related U.S. Application Data
(60) Provisional application No. 62/774,033, filed on Nov. 30, 2018.

(51) Int. Cl. G16H 40/67; G16H 10/60; G16H 50/20; G16H 20/00; G06N 5/04

(52) U.S. Cl. CPC G16H 40/67; G16H 10/60; G06N 5/04; G16H 20/00; G16H 50/20

(57) ABSTRACT

A personal health and wellness management system includes a communications module configured to: receive input data from a user device and/or a provider device, the input data being related to the health and/or wellness of a user, and provide output data to the user device and/or the provider device. A database is configured to store the input data. An insights module is configured to generate one or more insights based on the input data, and to provide the one or more insights as output data to the communications module.

### CROSS-REFERENCE TO RELATED APPLICATIONS

This application claims the benefit of U.S. Provisional Application No. 62/774,033, filed on Nov. 30, 2018, the entire contents of which are hereby incorporated by reference.

### FIELD OF THE INVENTION

The present invention relates to systems and methods for personal health management and the promotion of personal health and wellbeing.

### BACKGROUND OF THE INVENTION

Currently existing computer-based health and/or wellness platforms focus on singular aspects of health and/or wellness, such as, for example, meditation, online education, clinical diagnosis, telehealth visits, remote monitoring, and providing general health information. Some platforms use artificial intelligence to analyze user inputted data so as to recognize and predict potential health and/or wellness related concerns. However, these platforms create parallel and fragmented user experiences that are off-putting. In addition, these platforms are typically designed in accordance with the workflows of healthcare providers and systems, leading to further degradation of the user experience.

These platforms are also singularly tailored to western culture and western languages, such as, for example, American culture and the English language. As such, the user experience, and indeed the analyses, predictions and recommendations, provided by these platforms do not reflect the diversity of communities and societies served by these platforms. These platforms therefore tend to perpetuate healthcare disparities and inequities of care, which ultimately affects the health and wellbeing of those communities served. This is particularly problematic, as it is estimated that nearly 80% of health outcomes are influenced by happenings in the community or at home rather than in a hospital or clinic setting.

In order to overcome the above challenges, a personal health and wellness management system should provide self-guided therapeutic environment in a person-centric platform that accounts for the multi-lingual and multi-cultural nature of users. It should engage and interconnect users and providers to empower users to achieve their health and/or wellness goals via a truly holistic and inclusive platform that encourages self-care management.

In that regard, a system and method for personal health management is disclosed herein, which overcomes these and other shortcomings of prior systems and/or methods.

Other objects, advantages and novel features of the present invention will become apparent from the following detailed description of one or more preferred embodiments when considered in conjunction with the accompanying drawings. It should be recognized that the one or more examples in the disclosure are non-limiting examples and that the present invention is intended to encompass variations and equivalents of these examples. The disclosure is written for those skilled in the art. Although the disclosure use terminology and acronyms that may not be familiar to the layperson, those skilled in the art will be familiar with the terminology and acronyms used herein.

### BRIEF DESCRIPTION OF THE DRAWINGS

The features, objects, and advantages of the present invention will become more apparent from the detailed description, set forth below, when taken in conjunction with the drawings, in which like reference characters identify correspondingly throughout and wherein:

FIG. 1 is a schematic view of an exemplary personal health management system according to at least one embodiment;

FIG. 2 is a schematic view of a personal health management architecture according to at least one embodiment; and

FIG. 3 illustrates an exemplary method for managing personal health according to at least one embodiment.

### DETAILED DESCRIPTION OF THE DRAWINGS

The above described drawing figures illustrate aspects of the present invention in at least one embodiment, which is further defined in detail in the following description. Those having ordinary skill in the art may be able to make alterations and modifications to what is described herein without departing from its spirit and scope. While the present invention is susceptible of embodiment in many different forms, there is shown in the drawings and will herein be described in detail at least one embodiment of the invention with the understanding that the present disclosure is to be considered as an exemplification of the principles of the present invention, and is not intended to limit the broad aspects of the present invention to any embodiment illustrated. It will therefore be understood that what is illustrated is set forth for the purposes of example, and should not be taken as a limitation on the scope of the present invention.

As used herein, the terms “a” or “an” shall mean one or more than one. The term “plurality” shall mean two or more than two. The term “another” is defined as a second or more. The terms “including” and/or “having” are open ended (e.g., comprising). Reference throughout this document to “one embodiment”, “certain embodiments”, “an embodiment” or similar term means that a particular feature, structure, or characteristic described in connection with the embodiment is included in at least one embodiment of the present invention. Thus, the appearances of such phrases in various places throughout this specification are not necessarily all referring to the same embodiment.

Furthermore, the particular features, structures, or characteristics may be combined in any suitable manner on one or more embodiments without limitation. The term “or” as used herein is to be interpreted as inclusive or meaning any one or any combination.

In accordance with the practices of persons skilled in the art, the invention is described below with reference to operations that are performed by a computer system or a like electronic system. Such operations are sometimes referred to as being computer-executed. It will be appreciated that operations that are symbolically represented include the manipulation by a processor, such as a central processing unit, of electrical signals representing data bits and the maintenance of data bits at memory locations, such as in system memory, as well as other processing of signals. The memory locations where data bits are maintained are physical locations that have particular electrical, magnetic, optical, or organic properties corresponding to the data bits.

When implemented in software, the elements of the invention are essentially the code segments to perform the necessary tasks. The code segments can be stored in a processor readable medium. Examples of the processor readable mediums include an electronic circuit, a semiconductor memory device, a read-only memory (ROM), a flash memory or other non-volatile memory, a floppy diskette, a CD-ROM, an optical disk, a hard disk, etc.

In the following detailed description and corresponding figures, numerous specific details are set forth in order to provide a thorough understanding of the present invention. However, it should be appreciated that the invention may be practiced without such specific details. Additionally, for brevity sake well-known methods, procedures, components, and circuits have not been described in detail.

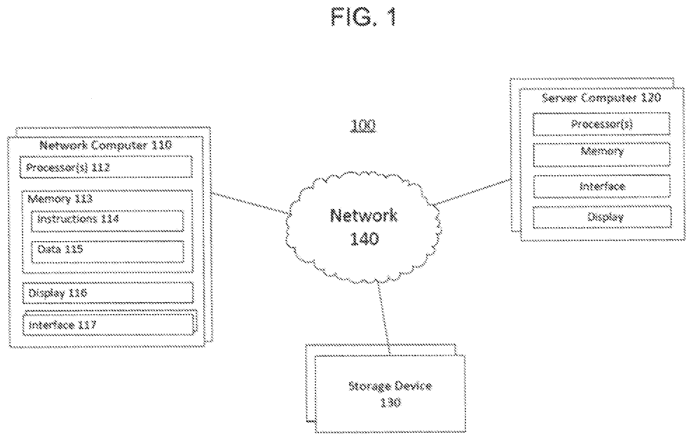

FIG. 1 illustrates an example personal health management system 100 in accordance with one or more aspects of the disclosure. In general, the personal health management system 100 provides an interactive online platform via which users may manage their personal health and/or wellness.

System 100 may include a plurality of computers and/or computing devices, such as, network computer(s) 110, server computer(s) 120, and storage device(s) 130. By way of example only, network computer(s) 110 is/are connected to network 140 and may include different types of components associated with a computer, such as one or more processors 112, memory 113, instructions 114, data 115, display 116, and an interface 117. Server computer(s) 120 may include server computers of health care service providers (e.g., hospitals, physicians, clinics, insurance companies, etc.), as well as sever computers hosting the interactive online platform.

The network computer(s) 110 include system and/or user computing devices, and may be mobile (e.g., laptop computer, tablet computer, smartphone, PDA, wearable, etc.) or stationary (e.g., desktop computer, etc.). In some aspects, the network computer(s) 110 include user devices configured to measure or otherwise track one or more aspects of the health and/or wellness of the associated user, and to communicate the measurements to the other computer devices on network 140.

The processor 112 of network computer 110 may instruct the components thereof to perform various tasks based on the processing of information and/or data that may have been previously stored or have been received, such as instructions 114 and/or data 115 stored in memory 113. The processor 112 may be a standard processor, such as a central processing unit (CPU), or may be a dedicated processor, such as an application-specific integrated circuit (ASIC) or a field programmable gate array (FPGA).

Memory 113 stores at least instructions 114 and/or data 115 that can be accessed by processor 112. For example, memory 113 may be hardware capable of storing information accessible by the processor, such as a ROM, RAM, hard-drive, CD-ROM, DVD, write-capable, read-only, etc. The set of instructions may be included in software that can be implemented on the network computer 110 and should be noted that the terms “instructions,” “steps,” “algorithm,” and “programs” may be used interchangeably. Data 115 can be retrieved, manipulated or stored by the processor 112 in accordance with the set of instructions 114 or other sets of executable instructions. The data 115 may be stored as a collection of data.

Accordingly, the network computer(s) 110 may include one or more software applications, stored in the memory 113, which software applications, when executed by the processor 110 configures the network computer(s) to function as described herein. In particular, the network computer(s) 110 may be configured to access the interactive online platform hosted by the server computer(s) 120, and to send and/or receive data to and from the server computer(s) 120. The software may further configure the network computer(s) 110 to allow the user to view and manage health and/or welfare information via interactive online platform.

The display 116 may be any type of device capable of communicating data to users, such as a liquid-crystal display (“LCD”) screen, a plasma screen, etc. Interface 117 allow users to communicate with the network computer 110 and may be a physical device (e.g., a port, a keyboard, a mouse, a touch-sensitive screen, microphone, camera, a universal serial bus (USB), CD/DVD drive, zip drive, card reader, etc.) and/or may be virtual (e.g., a graphical user interface “GUI,” etc.).

Server computer(s) 120 may also include one or more processors, memory, interface, and/or display and may be configured to communicate with other computer devices on network 140. The server computer(s) 120 may be rack mounted on a network equipment rack and/or located, for instance, in a data center. In one example, the server computer(s) 120 may use the network 140 to serve the requests of programs executed on network computer 110 and/or storage device 130.

The storage device 130 illustrated in FIG. 1 may be configured to store large quantities of data and/or information. For example, the storage device 130 may be a collection of storage components, or a mixed collection of storage components, such as ROM, RAM, hard-drives, solid-state drives, removable drives, network storage, virtual memory, cache, registers, etc. The storage device 130 may also be configured so that the network computer 110 and/or server computer 120 may access it via the network 140.

The network 140 may be any type of network, wired or wireless, configured to facilitate the communication and transmission of data, instructions, etc. from one component to another component of the network. For example, the network 140 may be a local area network (LAN) (e.g., Ethernet or other IEEE 802.03 LAN technologies), Wi-Fi (e.g., IEEE 802.11 standards, wide area network (WAN), virtual private network (VPN), global area network (GAN)), any combination thereof, or any other type of network.

It is to be understood that the network configuration illustrated in FIG. 1 serves only as an example and is thus not limited thereto. System 100, for instance, may include numerous other components connected to network 140, include more than one of each network component (as shown by the cascaded blocks), and network 140 may be connected to other networks.

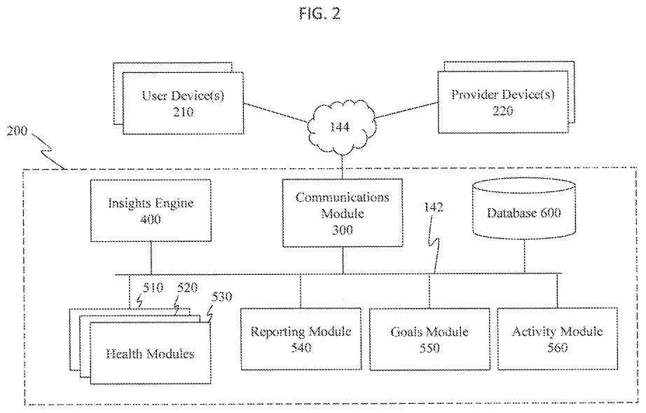

FIG. 2 illustrates a system architecture 200 for personal health and/or wellness management in accordance with at least one embodiment. The system architecture 200 includes a communications module 300, an insights engine 400, one or more support modules 510-560, and a database 600, each communicatively coupled via an internal network 142.

The internal network 142 may be any type of network configured to facilitate the communication and transmission of data, instructions, etc. from one component to another component of the network. The database 600 may be any type of database configured to store health and/or wellness related data that is used and/or generated by the respective modules. Each module and engine reflects hardware and/or software appropriately configured to perform the functions attributed to that module or engine, as described herein.

The communications module 300 is generally configured to manage the input/output of data and information between one or more user devices 210 and/or provider devices 220, on one other hand, and the support modules, database 600 and/or insights engine 400, on the other hand. In particular, the communications module 300 may be configured to provide one or more interactive graphical user interfaces for interacting with one or more interactive platforms provided, at least in part, by the one or more support modules. The communications module 300 may manage the user interfaces in accordance with user language and/or cultural preferences, which may be set via the support modules.

The communication module may be configured to recognize the format of input data and to convert the format of the input data such that the input data is usable by the other modules. For example, the communication module may utilize one or more of: language translation/recognition, speech-to-text conversion, image recognition, cultural recognition, augmentative/alternative communication (AAC) recognition, etc., to parse input data and to convert such input data into one or more data formats usable by the other modules.

The communication module may be configured to recognize the format of output data and to convert the format of the output data such that the output data is usable by the user and/or provider. The usable format may be consistent with one or more of: the user/provider device and/or user/provider preferences (e.g., language, culture, text vs. audio, etc.). For example, the communication module may utilize one or more of: language translation, speech generation, text generation, image generation, cultural reconstitution, provider system class and/or definition conversions, etc., to parse output data and to convert such output data into one or more data formats usable by the user and/or provider.

In at least one embodiment, the communications module 300 may accept both structured and unstructured input data as input data. Structured input data refers to data that is provided from a fixed field, has a defined data model, or is organized in a pre-defined manner known to the system. Unstructured input data refers to all other types of input data.

In at least one embodiment, the communications module 300 is configured to receive input data from electronic user devices that track health and/or wellness data. Such devices may include, for example, wearables, networked pace makers and other networked medical diagnostic devices.

The communications module 300 may further be configured to provide an interactive dashboard for display to the user and/or providers. The interactive dashboard may include user specific content and functions, in accordance with the various module functionalities.

The insights engine 400 is generally configured to analyze input data relating to user heath, and to provide output data, including wellness determinations and recommendations, and other insights, to the user and/or the provider. The insights engine 400 may utilize machine learning, neural networks and/or artificial intelligence to analyze the input data and to generate the insights therefrom. Input data may be provided, via the communications module 300, from the user and/or provider devices, and may be stored in the database 600. Moreover, input data may also include output data, including insights, that has been provided to the database 600.

Insights, as used herein, refer to identifications and/or predictions about potential health and/or wellness related issues. Such insights include, but are not limited to: identifications and/or predictions (including warnings) of physical and/or mental health, safety and/or wellness risks. Insights may also refer to recommendations, including but not limited to: recommended actions for mitigating identified and/or predicted risk. Insights may be generated according to various scales or associations, including but not limited to: individual, family and/or social groupings; occupational characteristics (e.g., company, industry, job title, etc.); cultural groupings; and large-scale (e.g., global) healthcare ecosystem.

In at least one embodiment, the insights engine 400 may be configured to determine, based on the input data, language and/or cultural factors relevant to generating insights. The insights engine 400 may also be configured to utilize such language and/or cultural factors in generating insights.

In at least one embodiment, the insights engine 400 may be configured to determine, based on the input data, relationship factors relevant to generating insights. The insights engine 400 may also be configured to utilize such relationship factors in generating insights. Such relationship factors may include, for example, familial or other social relationships among users.

In at least one embodiment, the insights engine 400 may be configured to detect trends and/or patterns in one or more sets of input data, e.g., the input data related to a user. The insights engine 400 may also be configured to utilize such detected trends and/or patterns in generating insights. The detected trends and/or patterns may relate to, for example, health history, personal habits and/or goals, social and environmental determinants, at-risk behaviors, and mental and behavioral determinants. The trends and/or patterns can be detected based on one or more determined factors, including but not limited to: language and/or cultural factors, and relationship factors.

In at least one embodiment, the insights engine 400 may be configured to predict health and/or wellness related conditions. The insights engine 400 may also be configured to utilize such predictions in generating additional insights, e.g., recommendations.

In operation, the insights engine 400 is configured to identify health and/or wellness related issues, behaviors and goals of the user, and to provide recommendations based on the identified health and/or wellness related issues.

For example, the insights engine 400 may identify, as the health and/or wellness related condition, that the user has influenza, based on analysis of biometric data entered by the user, e.g., temperature and other symptoms, as well as the insight that the user lives in a geographic area where influenza is currently spreading, and the insight that the user's spouse was recently diagnosed with influenza. This insight (i.e., the user has influenza) may then be stored in the database 600, via a user profile, as data related to the health and/or wellness information of the user.

The insights engine 400 may further identify, as the health and/or wellness related goal, that the user has a goal of recovering from influenza, and therefore requires a minimum number of hours of sleep and should not exercise. The insight is preferably based, at least in part, on one or more of: the language/cultural and relationship factors of the user. For example, where cultural or other factors may indicate an ideal minimum for hours of sleep is not realistically attainable or necessary, the minimum for hours of sleep may be modified from the ideal. The insights engine 400 may thereafter generate a recommendation of the minimum number of hours of sleep without exercise. This recommendation may also be stored as health and/or wellness data related to the user.

The insights engine 400 may still further determine, as the health and/or wellness behavior, that the user is not sleeping the minimum number of hours and is exercising heavily. The determination may be made from a comparison of input data, e.g., biometric data from a linked smart device, with the recommendation insight. This determination may also be stored as health and/or wellness data related to the user.

As such, the insights engine 400 reflects an evolving understanding of the user's current health and/or wellbeing, including risks, goals, recommendations, etc.

In at least one embodiment, the insights engine 400 is configured to provide a request data object, the acceptance of which changes a third-party system in some way. The insights engine 400 may be configured to send a request to the user to, for example, add/delete/change a calendar entry on the user's digital calendar, set a reminder on the user device 210, cancel a subscription, enroll in a class or activity, etc.

The one or more support modules are generally configured to provide interactive platforms for receiving and/or outputting health and/or wellness related data and information.

A personal health module 510 is generally configured to provide an interactive platform for receiving and/or outputting personal health and/or wellness related data and information of the user. The personal health module 510 may be configured such that the user and/or provider may provide input data related to the personal health and/or wellness of the user. The input data may be provided as structured and/or unstructured data. The personal health module 510 may utilize forms, questionnaires, checklists, and other techniques for gathering the input data.

For example, the personal health module 510 may present, via the communications module 300, one or more questions about the heath and/or wellness of the user. The presentation may be textual, auditory, etc. The one or more questions may be prioritized according to existing and developing insights—and therefore the prioritization may be dynamically determined by the responses. Responses to the questions may be provided, via the communications module 300, as structured and/or unstructured data—and may be textual, auditory, etc. The questions may be presented to the user and/or providers, and responses may be receive by the user and/or providers. The heath and/or wellness related information gathered, or aspects thereof or insights therefrom, may be displayed via the personal heath module platform.

The personal health and/or wellness related data for the user may be stored in the database 600, and may be retrieved by the personal health module 510 to generate personal health and/or wellness related content for display via the interactive platform of the personal health module 510. Insights may also be retrieved by the personal health module 510 to generate personal health and/or wellness related content for display via the interactive platform. Such content may include, for example, health and/or wellness related models, charts, analysis, etc.

A family health module 520 is generally configured to provide an interactive platform for receiving and/or outputting personal health and/or wellness related data and information of the user's family. The family health module 520 may be configured such that the user and/or provider may provide input data related to the personal health and/or wellness of members of the user's family. The input data may be provided as structured and/or unstructured data. The family health module 520 may utilize forms, questionnaires, checklists, and other techniques for gathering the input data.

Users may become associated as family members via existing social media techniques, such as the utilization of “friend requests” and the like. Accordingly, the family health module 520 may be configured to provide a quasi-social media platform that keeps track of the other-user-family-network of the user.

The personal health and/or wellness related data for the user's family may be stored in the database 600, and may be retrieved by the family health module 520 to generate family health and/or wellness related content for display via the interactive platform of the family health module 520. Insights may also be retrieved by the family health module 520 to generate family health and/or wellness related content for display via the interactive platform. Such content may include, for example, health and/or wellness related models, charts, analysis, etc.

A community health module 530 is generally configured to provide an interactive platform for receiving and/or outputting personal health and/or wellness related data and information of the user's community. The community health module 530 may be configured such that the user and/or provider may provide input data related to the personal health and/or wellness of members of the user's community. The input data may be provided as structured and/or unstructured data. The family health module 520 may utilize forms, questionnaires, checklists, and other techniques for gathering the input data.

Users may become associated as members of a community via existing social media techniques, such as the utilization of “friend requests” and the like. Accordingly, the community health module 530 may be configured to provide a quasi-social media platform that keeps track of the other-user-network of the user.

The personal health and/or wellness related data for the user's community may be stored in the database 600, and may be retrieved by the community health module 530 to generate community health and/or wellness related content for display via the interactive platform of the community health module 530. Insights may also be retrieved by the community health module 530 to generate community health and/or wellness related content for display via the interactive platform. Such content may include, for example, health and/or wellness related models, charts, analysis, etc.

The various health modules may further support the interactive dashboard having user specific content and functions for providing the input data, as well as for displaying insights and other output data and information.

A reporting module 540 is generally configured to provide an interactive platform for tracking health and/or wellness parameters and information.

In operation, the reporting module 540 maintains a record, via a user profile stored in the database 600, of the health and/or wellness related data and insights associated with the user. This information can include, for example, vital statistics, medical test results, mental evaluation results, exercise habits, work habits, cultural factors, at-risk behaviors, sleep statistics, and any other data relevant to the health and/or wellness of the user.

The insights engine 400, as well as the other modules, may access the health and/or wellness related data and insights associated with the user via the user profile, so as to utilize the information to provide insights and otherwise provide module functionality. In particular, the insight engine may tracks user activity, time spent on the platform, and features used so as to utilize such information to generate further insights.

The reporting module 540 may also be configured to convert health and/or wellness data and/or insights into a report that may be provided to the user and/or providers via the communications module 300. The reports may relate to personal, family and/or community health and/or wellness, and may include data and/or insights related to, for example, preventative care, learning readiness, self-care management, lifestyle and habits, coping and support, immunizations, health experience, family medical histories and trends, goal progress, recommendations, etc.

The reporting module 540 may also support the interactive dashboard having user specific content and functions, which may in turn provide access to the reports. The reports may be provided in any known format. Moreover, the reports may be used in research studies. Accordingly, the reporting module 540 may be configured to allow users opt-in or opt-out of such research studies.

A goals module 550 is generally configured to provide an interactive platform for tracking and managing user goals and progress towards those goals. This may include, for example, maintaining and tracking milestones towards one or more goals.

In operation, the goals module 550 receives user goals and goal-progress information as input data, which may be provided by the user and/or providers as input data. In some embodiments, the goals may be suggested as insights. For example, the user may provide unstructured data describing their vision, purpose, obstacles and resources, from which the insights engine 400 may determine a goal and one or more milestones. Alternatively, the user may select a goal and/or milestones from among a plurality of predetermined goals and/or milestones. Goal progress may be tracked by monitoring the input data to determine, via the insights engine 400, whether the user has met set goals and/or reached set milestones.

The goals module 550 may further have access to a digital calendar of the user, which may be modified in accordance with the goals and/or milestones. In some embodiments, activities in accordance with the goals and/or milestones may be added to the calendar, removed from the calendar, or modified within the calendar. The activities may be provided as input data, structured or unstructured, via the communications module 300.

The goals module 550 may further track the progress of the user towards set goals based on an analysis, via the insight engine, of input data. For example, the goals module 550, via the insights engine 400 analysis of the input data, may determine that the user has missed an unacceptable number of days of a scheduled activity whose completion is towards a set goal. The goals module 550 may accordingly track the goal as not being progressed towards—and the insights engine 400 may optionally provide a notification or warning to the user, via the communications module 300, of the lack of progress. Such notification may be provided by way of the personal health module 510 asking questions about the heath and/or wellness of the user, e.g., “I see you have missed several yoga classes. Is everything okay?” The responsive input data can then be utilized by the insights engine 400 to generate further insights. Similar inquiries and/or notifications may be provided to providers, e.g., doctors, directing the providers to, e.g., follow up with the user and/or the user profile.

The goals module 550 may further support the interactive dashboard having user specific content and functions, which may in turn provide access to goals and progress management and tracking.

An activity module 560 is generally configured to provide an interactive platform that tracks and manages participation in health and/or wellness related activities and meetings. The functionality of the activity module 560 in several respects is similar to that of the goals module 550, except for health and/or wellness related activities and meetings, which may be related to goals or milestones, but that are not themselves goals or milestones.

In some embodiments, the activity module 560 may manage and track user participation in health and/or wellness related activities. Such activities may include, for example, therapy activities (e.g., journaling, art, music, healing touch, yoga, biofeedback, tai chi, augmented reality, etc.), exercise activities (e.g., pilates, running, cardio-kickboxing, zumba, etc.), and educational activities (e.g., breastfeeding courses, immunization education, sexual education, etc.). The activities may be in-person, remote, live and/or on-demand. For remote on-demand activities, the database 600 may store a library of such activities, preferably in audio/visual format, that are accessible to the user via the activity module 560.

The activity module 560 may further have access to the digital calendar of the user, which may be modified in accordance with selected activities. In some embodiments, the activities may be added to the calendar, removed from the calendar, or modified within the calendar. The activities may be provided as input data, structured or unstructured, via the communications module 300. Moreover, similar to the goals module 550, the activity module 560 may further track the progress of the user towards completion of selected activities based on an analysis, via the insight engine, of input data.

In some embodiments, the activity module 560 may manage and track meetings and appointments with heath and/or wellness providers (e.g., physician, psychiatrist, urologist, cardiologist, etc.) as a particular type of activity. As with other types of heath and/or wellness related activities, such meetings may be tracked and managed via use of the calendar and input data, and insights may be generated based thereon.

Each of the aforementioned modules may further provide for the sharing of user heath and/or wellness related data and/or content provided thereon with providers and/or other users (e.g., family or community members). Each of the modules may further provide for the sharing of user heath and/or wellness related data and/or content provided thereon with providers and/or other users (e.g., family or community members).

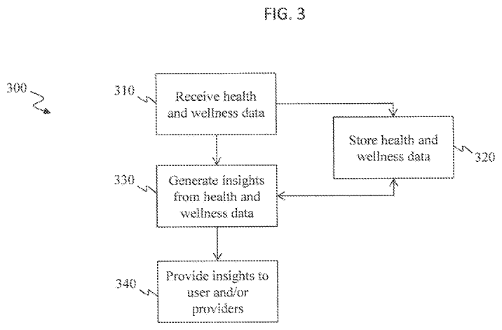

An exemplary method 30 for managing personal health and/or wellness in accordance with at least one embodiment will now be described with reference to FIG. 3. At step 310, health and/or wellness related information is input to the system 100 as input data, as described herein. At step 320, the input data is stored in the database 600 in association with the user profile, as described herein. At step 330, the insights engine 400 generates one or more insights based on the information stored in the database 600, which insights are stored in the database 600 600 as additional input data. At step 340, the generated insights related to the user are provided to the user and/or providers.

The embodiments described in detail above are considered novel over the prior art and are considered critical to the operation of at least one aspect of the described systems, methods and/or apparatuses, and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus, if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense, it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

Reference throughout this document to “one embodiment”, “certain embodiments”, “an embodiment” or similar term means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner on one or more embodiments without limitation.

In accordance with the practices of persons skilled in the art of computer programming, the invention is described herein with reference to operations that are performed by a computer system or a like electronic system. Such operations are sometimes referred to as being computer-executed. It will be appreciated that operations that are symbolically represented include the manipulation by a processor, such as a central processing unit, of electrical signals representing data bits and the maintenance of data bits at memory locations, such as in system memory, as well as other processing of signals. The memory locations where data bits are maintained are physical locations that have particular electrical, magnetic, optical, or organic properties corresponding to the data bits.

Furthermore, the operations described herein may be implemented via hardware, software, firmware or any combination thereof, unless expressly indicated otherwise. If implemented in software, the operations may be stored in a memory as one or more instructions on a computer readable medium, including any available media accessible by a computer that can be used to store desired program code in the form of instructions, data structures or the like. Thus, certain aspects may comprise a computer program product for performing the operations presented herein, such computer program product comprising a computer readable medium having instructions stored thereon, the instructions being executable by one or more processors to perform the operations described herein. It will be appreciated that software or instructions may also be transmitted over a transmission medium as is known in the art. Further, modules, engines, logic and/or other appropriate means for performing the operations described herein may be utilized in implementing the operations described herein.

In accordance with the descriptions herein, the term “server” means a functionally-related group of electrical components, such as a computer system that may or may not be connected to a network and which may include both hardware and software components, or alternatively only the software components that, when executed, carry out certain functions. The “server” may be further integrated with a database management system and one or more associated databases.

The term “computer readable medium” refers to any non-transitory media that participates in providing instructions to a processor for execution. Such a non-transitory medium may take many forms, including but not limited to volatile and non-volatile media. Non-volatile media includes, for example, optical or magnetic disks. Volatile media includes dynamic memory for example and does not include transitory signals, carrier waves, or the like.

The terms “module” and “engine” refer to hardware, firmware, software in execution on a machine, and/or combinations of each to perform a function(s) or an action(s), and/or to cause a function or action to occur from another module, engine, method, and/or system. The terms may include a software controlled microprocessor, a discrete logic (e.g., ASIC), an analog circuit, a digital circuit, a programmed logic device, a memory device containing instructions, and so on. This may also include one or more gates, combinations of gates, or other circuit components.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. A personal health and wellness management system, the system comprising:
a communications module configured to:
receive input data from a user device and/or a provider device, the input data being related to the health and/or wellness of a user, and
provide output data to the user device and/or the provider device;
a database configured to store the input data; and
an insights module configured to generate one or more insights based on the input data, and to provide the one or more insights as output data to the communications module.

2. The personal health and wellness management system of claim 1, wherein the insights module generates insights based at least in part on input data reflecting a culture of the user.

3. The personal health and wellness management system of claim 1, wherein the insights module generates insights based at least in part on input data reflecting a family of the user.

4. The personal health and wellness management system of claim 1, wherein the insights module generates insights based at least in part on input data reflecting a community of the user.

5. The personal health and wellness management system of claim 1, wherein the insights module is further configured to provide the one or more insights as input data to the database.

6. The personal health and wellness management system of claim 1, wherein the one or more insights identify health and/or wellness related conditions of the user.

7. The personal health and wellness management system of claim 1, wherein the one or more insights include recommendations for improving and/or managing the health and/or wellness of the user.

8. The personal health and wellness management system of claim 1, further comprising:
a goals module configured to manage one or more user health and/or wellness goals, and to track user progress towards the one or more user health and/or wellness goals.

9. The personal health and wellness management system of claim 8, wherein the goals module tracks user progress based on the input data.

10. The personal health and wellness management system of claim 1, further comprising:
an activity module configured to manage one or more health and/or wellness related activities, and to track user participation in the one or more health and/or wellness activities.

11. The personal health and wellness management system of claim 9, wherein the activity module tracks user participation based on the input data.

12. A personal health and wellness management method, the method comprising:
receiving input data from a user device and/or a provider device, the input data being related to the health and/or wellness of a user;
storing the input data in a database;
generating one or more insights based on the input data;
providing the one or more insights as output data to the user device and/or the provider device.

13. The method of claim 12, wherein the insights are generated based at least in part on input data reflecting a culture of the user.

14. The method of claim 12, wherein the insights are generated based at least in part on input data reflecting a family of the user.

15. The method of claim 12, wherein the insights are generated based at least in part on input data reflecting a community of the user.

16. The method of claim 12, further comprising:
providing the one or more insights as input data to the database.

17. The method of claim 12, wherein the one or more insights identify health and/or wellness related conditions of the user.

18. The method of claim 12, wherein the one or more insights include recommendations for improving and/or managing the health and/or wellness of the user.
